(19) United States
(12) Patent Application Publication
(10) Pub. No.: US 2026/0286402 A1
(43) Pub. Date: Sep. 24, 2026

(54) PROCESS FOR THE PRODUCTION OF BIO-OIL COUPLING MICROWAVE HYDROLYSIS PRETREATMENT OF BIOMASS WASTE AND THE FERMENTATION OF MICROORGANISMS

(71) Applicant: The Board of Trustees of the Leland Stanford Junior University, Stanford, CA (US)

(72) Inventors: Gabriella Alina DWECK, Palo Alto, CA (US); Kelly Lynn REDMOND, San Francisco, CA (US)

(21) Appl. No.: 19/477,963

(22) PCT Filed: May 6, 2024
(86) PCT No.: PCT/US2024/028032

§ 371 Date: Oct. 23, 2025

Related U.S. Application Data
(60) Provisional application No. 63/501,612, filed on May 11, 2023.

(51) Int. Cl. C12P 7/6409; C08J 3/28; C12N 1/165; C12R 1/645

(52) U.S. Cl. CPC C12P 7/6409; C08J 3/28; C12N 1/165; C12P 2203/00; C12R 2001/645

(57) ABSTRACT

A method for producing bio-oil using cellulosic or lignocellulosic biomass as an input is provided. Microwave hydrolysis of the cellulosic or ignocellulosic biomass is used as a pretreatment method to extract fermentable sugars from the cellulosic or lignocellulosic biomass. An oleaginous yeast is fermented using the extracted fermentable sugars to produce a bio-oil.

### CROSS-REFERENCE TO RELATED APPLICATION

This application claims the benefit of priority of U.S. Application No. 63/501,612, filed May 11, 2023, which is incorporated herein by reference for all purposes.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

This invention relates to the production of bio-oils. More specifically the invention relates to the production of bio-oils derived from biomass waste.

### SUMMARY

To achieve the foregoing and in accordance with the purpose of the present disclosure, a method for producing bio-oil using cellulosic or lignocellulosic biomass as an input is provided. Microwave hydrolysis of the cellulosic or lignocellulosic biomass is used as a pretreatment method to extract fermentable sugars from the cellulosic or lignocellulosic biomass. An oleaginous yeast is fermented using the extracted fermentable sugars to produce a bio-oil.

These and other features of the present disclosure will be described in more detail below in the detailed description and in conjunction with the following figures.

### BRIEF DESCRIPTION OF THE DRAWINGS

The present disclosure is illustrated by way of example, and not by way of limitation, in the figures of the accompanying drawings and in which like reference numerals refer to similar elements and in which:

FIG. 1 depicts a high level flow chart describing a method of processing biomass in various embodiments.

In the drawings, like reference numerals are sometimes used to designate like structural elements. It should also be appreciated that the depictions in the figures are diagrammatic and not to scale.

### DETAILED DESCRIPTION OF EMBODIMENTS

This invention relates to the production of bio-oils, derived from biomass waste in which cellulosic or lignocellulosic biomass is valorized by extracting fermentable sugars via microwave hydrolysis and used as a fermentation substrate for oleaginous yeast. The technology can be broken down into four main components: preprocessing biomass sources in preparation for hydrolysis, pretreatment of biomass using single-step microwave hydrolysis technology, fermentation of the oleaginous yeast Lipomyces starkeyi to accumulate intracellular lipids, and lipid extraction to yield a sustainable bio-oil with properties similar to palm oil.

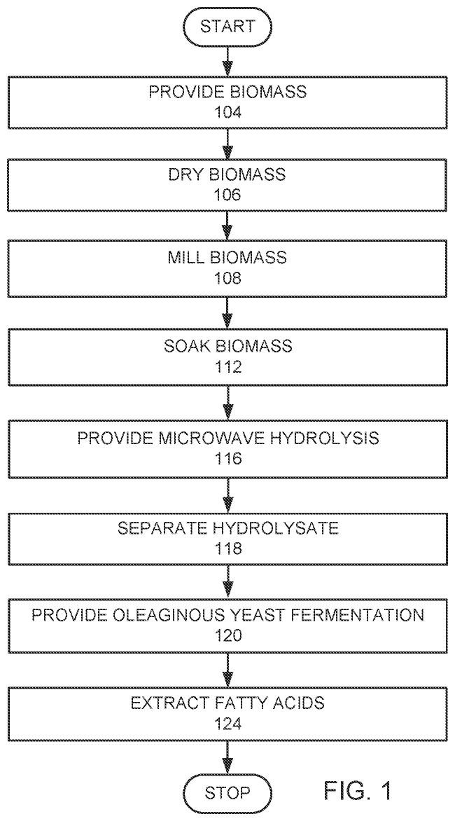

In order to facilitate understanding, FIG. 1 is a high level flow chart of a method that may be used in some embodiments. In such embodiments, biomass is provided (step 104). In some embodiments, the biomass comprises at least one of forest residues, agricultural waste, food waste, and macroalgae.

The biomass is dried (step 106). In some embodiments, at least one of the sun, a heater, a drying oven, and forced air may be used to dry the biomass.

Next, the biomass is milled (step 108). In some embodiments, the biomass is milled into particles with a length of less than 5 mm. In some embodiments, the biomass is milled into particles with a length of less than 3 mm. In some embodiments, the biomass is milled into particles with a length of less than 2 mm. In some embodiments, the biomass is milled into particles with a length in the range of 1 to 2 mm.

The milled biomass particles are then soaked or presoaked (step 112). In some embodiments, the milled biomass particles are soaked in water, such as deionized water. In some embodiments, the milled biomass particles are soaked in deionized water with a loading rate in the range of 0.5% to 40% by weight of solid mass to water. In some embodiments, the milled biomass particles are soaked in deionized water with a solid loading rate in the range of 1% to 20% by weight. In some embodiments, the milled biomass particles are soaked in deionized water with a loading rate in the range of 3% to 10% by weight. For example, a 5% loading rate would provide 40 milliliters of water for 2 grams of biomass material. In some embodiments, the soaking is maintained for a period of 0 to 48 hours. In some embodiments, the soaking is maintained for a period of 6 to 36 hours.

The soaked biomass is then subjected to a microwave hydrolysis process (step 116). In some embodiments, the microwave hydrolysis is enzyme free and acidic additive free. In some embodiments, the microwave hydrolysis heats the biomass to a temperature of at least 100° C. In some embodiments, the microwave hydrolysis heats the biomass to a temperature of at least 120° C. In some embodiments, the microwave hydrolysis heats the biomass to a temperature of at least 130° C. In some embodiments, the microwave hydrolysis heats the biomass to a temperature of at least 220° C. In some embodiments, the microwave hydrolysis is provided for a time in the range of 30 seconds to 50 minutes. In some embodiments, the microwave hydrolysis is provided for a time in the range of 1 minute to 20 minutes.

The microwave hydrolysis process (step 116) is a single-step, additive-free method of valorizing recalcitrant biomass by breaking down glycosidic bonds to extract fermentable sugars from cellulosic or lignocellulosic waste. In the microwave hydrolysis process, the soaked biomass is loaded into a microwave chamber and heated via microwave energy to extract monosaccharides from the cellulose and hemicellulose fractions of the biomass within a biomass slurry. The biomass slurry created by the microwave hydrolysis process comprises a sludge waste stream that can be easily reintegrated into the environment and an aqueous solution of pentose and hexose fermentable sugars called a cellulosic or lignocellulosic hydrolysate. This hydrolysate is an intermediate product and is leveraged in the second process step as a fermentation substrate.

The microwave hydrolysis process (step 116) utilizes additive-free, single-step microwave hydrolysis because of its economic viability, downstream processing benefits, increased glucose yield, and success with a diverse range of biomass sources. Currently, the chief energetic demand in processing biomass is the heating and electrical power for pre-treatment. By removing the need for enzymes and using a more efficient microwave heating source, the microwave hydrolysis process can reduce the cost of biomass processing by up to 30%. The lack of hazardous enzymatic and acidic additives in the microwave hydrolysis process will allow the waste streams to be easily reintegrated into the environment, potentially as a beneficial byproduct, such as compost, fertilizer, or biochar.

The biomass slurry is separated into sludge waste in a solid phase and hydrolysate in a liquid phase (step 118). In some embodiments, at least one of a centrifuge, decanter, and press is used to separate the solid phase from the hydrolysate. In some embodiments, the hydrolysate comprises carbohydrates.

The hydrolysate is then provided to an oleaginous yeast fermentation process (step120). The fermentation process leverages the biomass-based hydrolysate as a substrate to grow a wild-type oleaginous yeast strain, which produces intracellular lipids that ultimately serve as Oleo's sustainable bio-oil product. In some embodiments, during the oleaginous yeast fermentation process (step120), a fermentation broth consisting of hydrolysate and enriched media components is loaded into a bioreactor and inoculated with oleaginous yeast. Over a 5-day period, the yeast consumes the pentose and hexose sugars in the hydrolysate, multiply, and accumulate up to 70% of their cell weight in intracellular lipids with a fatty-acid profile similar to conventional palm oil.

In some embodiments, the oleaginous yeast fermentation process (step120) uses Lipomyces starkeyi as the yeast strain for fermentation to optimize for lipid accumulation. Lipomyces starkeyi is an oleaginous yeast that has been studied extensively for lipid accumulation. Lipomyces starkeyi has the potential to accumulate more than 70% of its biomass as intracellular lipids using a mixture of hexose and pentose sugar and is resilient against common inhibitory compounds such as vanillin, furfural, and hydroxymethylfurfural (HMF).

After fermentation is completed, fatty acids are extracted (step 124). In some embodiments, the resultant lipids are extracted via liquid-liquid phase separation (or liquid-liquid extraction) and centrifugation. In some embodiments, the resultant lipids are extracted via microwave assisted extraction. The resultant lipid is composed of long-chain fatty acids, which are primarily palmitic, oleic, stearic, and linoleic acids. Generally, the long-chain fatty acids have more than 12 carbons These results are promising for an application as a drop-in replacement for palm oil in the biofuel sector as biofuel favors a higher percentage of monounsaturated fatty acids, the chief of which is oleic, for their liquidity at room temperature.

Commercial applications of this technology include drop-in replacements for seed oils including palm oil and petroleum derivatives in the consumer goods industry for use in personal care products, cleaning agents, beauty products, and processed foods, and in the biofuels industry as a feedstock for second generation biofuels with applications in marine shipping and aviation fuels.

Some of the embodiments have several advantages. First, some embodiments can combine multiple feedstock sources to circumvent traditional supply chain issues surrounding the production of second and third generation products such as seasonality. Second, some embodiments can produce a synthetic alternative at roughly two-thirds the cost of traditional fermentation methods by using repurposed biomass waste streams as feedstock, rather than sugar cane, and using a novel thermomechanica pretreatment method that offers increased efficiency. In some embodiments, other biomass besides oil palm fronds may be used. Microwave hydrolysis is used to extract sugars from the plant material. In some embodiments, the microwave hydrolysis is at least one of enzyme free and acidic additive free. In some embodiments, other oleaginous yeast may be used instead of Lipomyces starkeyi to ferment the sugar to produce lipids. The lipids comprise fatty acids comprising oleic acid and palmitic acids serving as a sustainable replacement for palm oil or intermediate bio-oil for biofuel production.

While this invention has been described in terms of several preferred embodiments, there are alterations, permutations, modifications, and various substitute equivalents, which fall within the scope of this invention. It should also be noted that there are many alternative ways of implementing the methods and apparatuses of the present invention. It is therefore intended that the following appended claims be interpreted as including all such alterations, permutations, modifications, and various substitute equivalents as fall within the true spirit and scope of the present invention. As used herein, the phrase “A, B, or C” should be construed to mean a logical (“A OR B OR C”), using a non-exclusive logical “OR,” and should not be construed to mean ‘only one of A or B or C. Each step within a process may be an optional step and is not required. Different embodiments may have one or more steps removed or may provide steps in a different order. In addition, various embodiments may provide different steps simultaneously instead of sequentially.

1. A method for producing bio-oil using cellulosic or lignocellulosic biomass as an input, comprising the steps of:
a) using microwave hydrolysis of the cellulosic or lignocellulosic biomass as a pretreatment method to extract fermentable sugars; and
b) fermenting an oleaginous yeast using the extracted fermentable sugars to produce a bio-oil.

2. The method, as recited in claim 1, wherein the oleaginous yeast comprises Lipomyces starkeyi.

3. The method, as recited in claim 1, wherein fermenting the oleaginous yeast uses a fermentation substrate of cellulosic or lignocellulosic hydrolysate produced by the microwave hydrolysis.

4. The method, as recited in claim 1, further comprising extracting lipids from the oleaginous yeast.

5. The method, as recited in claim 1, further comprising milling the cellulosic or lignocellulosic biomass.

6. The method, as recited in claim 1, further comprising drying the cellulosic or lignocellulosic biomass.

7. A method, as recited in claim 1, wherein the bio-oil comprises long-chain fatty acids.

8. The method, as recited in claim 1, wherein the using the microwave hydrolysis is enzyme free and acidic additive free.

9. The method, as recited in claim 1, wherein using the microwave hydrolysis heats the cellulosic or lignocellulosic biomass to a temperature of at least 120° C. for a time in a range of 1 to 20 minutes.

10. The method, as recited in claim 1, further comprising:
milling the cellulosic or lignocellulosic biomass to particles with a length of less than 2 mm; and
presoaking the particles in water at a loading rate between 1% and 20% by weight of solid mass to water prior to using the microwave hydrolysis.

11. The method, as recited in claim 1, wherein using the microwave hydrolysis of the cellulosic or lignocellulosic biomass as a pretreatment method to extract fermentable sugars creates biomass slurry, and further comprising centrifuging biomass slurry to separate the biomass slurry into a liquid phase and a solid phase, wherein the liquid phase is a hydrolysate used as a substrate for the oleaginous yeast fermentation.

12. The method, as recited in claim 11, wherein the hydrolysate comprises carbohydrates.

13. The method, as recited in claim 1, wherein lipids are extracted from oleaginous yeast via at least one of microwave and liquid-liquid extraction.

14-24. (canceled)
